(19) United States
(12) Patent Application Publication
(10) Pub. No.: US 2024/0055153 A1
(43) Pub. Date: Feb. 15, 2024

(54) MULTICORE CABLE AND MULTICORE CABLE ASSEMBLY

(71) Applicant: Proterial, Ltd., Tokyo (JP)

(72) Inventors: Atsuro YAGUCHI, Tokyo (JP); Takanobu WATANABE, Tokyo (JP)

(21) Appl. No.: 18/231,042

(22) Filed: Aug. 7, 2023

(30) Foreign Application Priority Data
Aug. 12, 2022 (JP) 2022-128637

(51) Int. Cl. H01B 7/00; A61B 5/00

(52) U.S. Cl. CPC H01B 7/0009; A61B 2562/222; A61B 5/6852

(57) ABSTRACT

A multicore cable is provided with a plurality of insulated electric wires, each including a metal conductor wire and an insulating layer covering the metal conductor wire. The plurality of insulated electric wires are twisted together, and each of the plurality of insulated electric wires is plastically stretched in a longitudinal direction at an elongation rate of 0.5% or more and 10.0% or less. A multicore cable assembly is provided with the multicore cable, and a terminal member to which the plurality of insulated electric wires are connected at an end of the multicore cable.

### CROSS-REFERENCE TO RELATED APPLICATIONS

The present patent application claims the priority of Japanese patent application No. 2022-128637 filed on Aug. 12, 2022, and the entire contents thereof are hereby incorporated by reference.

### TECHNICAL FIELD

The present invention relates to a multicore cable in which a plurality of insulated electric wires (i.e., insulation-coated wires) are twisted together, and a multicore cable assembly including the multicore cable.

### BACKGROUND OF THE INVENTION

Conventionally, a multicore cable in which a plurality of insulated electric wires are twisted together is used as a catheter cable for medical equipment, for example. The present applicant has proposed the multicore cables described in Patent Literatures 1 and 2 as such multicore cables.

### CITATION LIST

Patent Literature 1: JPH9-219115A
Patent Literature 2: JPH10-134649A

### SUMMARY OF THE INVENTION

In recent years, the number of core wires in a catheter cable has tended to increase due to the sophistication of medical equipment. On the other hand, from the viewpoint of reducing the burden on the subject, etc., there is a demand for reducing the outer diameter of the cable. For this reason, as the core wire, a super fine (i.e., ultra-thin) conductor having a diameter of, e.g., less than 0.1 mm has been used. However, when such super fine core wires are used, the core wires have a bending tendency, and each core wire bends irregularly at the ends of the cable. In some cases, the workability of terminal processing is remarkably lowered.

The present invention has been made in view of the above circumstances, and an object of the present invention is to provide a multicore cable and a multicore cable assembly using a plurality of insulated electric wires as core wires, which can reduce the bending tendency of the insulated electric wires and improve the workability of terminal processing.

In order to solve the above-mentioned problems, the present invention provides a multicore cable, comprising:
a plurality of insulated electric wires, each comprising a metal conductor wire and an insulating layer covering the metal conductor wire,
wherein the plurality of insulated electric wires are twisted together,
wherein each of the plurality of insulated electric wires is plastically stretched in a longitudinal direction at an elongation rate of 0.5% or more and 10.0% or less.

Further, in order to solve the above problems, the present invention provides a multicore cable assembly, comprising:
the multicore cable as described above; and
a terminal member to which the plurality of insulated electric wires are connected at an end of the multicore cable.

### Effect of the Invention

According to the multicore cable and the multicore cable assembly of the present invention, it is possible to reduce the bending tendency of the insulated electric wires and improve the workability of terminal processing of the multicore cable.

### BRIEF DESCRIPTION OF DRAWINGS

FIG. 1A is an explanatory diagram showing a state of use of a multi-electrode catheter in which a multicore cable according to an embodiment of the present invention is used as a catheter cable.

FIG. 1B is a cross-sectional view of the catheter cable.

FIG. 2A is an explanatory diagram showing the end of the catheter cable before a plurality of insulated electric wires are connected to a substrate.

FIG. 2B is an explanatory diagram showing a state where a plurality of insulated electric wires of the catheter cable are connected to the substrate.

FIG. 3 is an explanatory diagram showing, as a comparative example, an example of a plurality of insulated electric wires at the end of the catheter cable when the metal conductor wires are not plastically stretched in the longitudinal direction.

FIG. 4A is an explanatory diagram showing an enlarged portion A of FIG. 3.

FIG. 4B is a cross-sectional view of the insulated electric wire along a line B-B of FIG. 4A.

FIG. 5 is an explanatory diagram showing a configuration example of a manufacturing apparatus for manufacturing a wire bundle by twisting a plurality of insulated electric wires.

FIG. 6 is a photograph showing a catheter cable according to an example.

FIG. 7 is a photograph showing a plurality of insulated electric wires that are not plastically stretched and a binder tape wrapped around the outer circumferences of the plurality of insulated electric wires.

### DETAILED DESCRIPTION OF THE INVENTION

### Embodiment

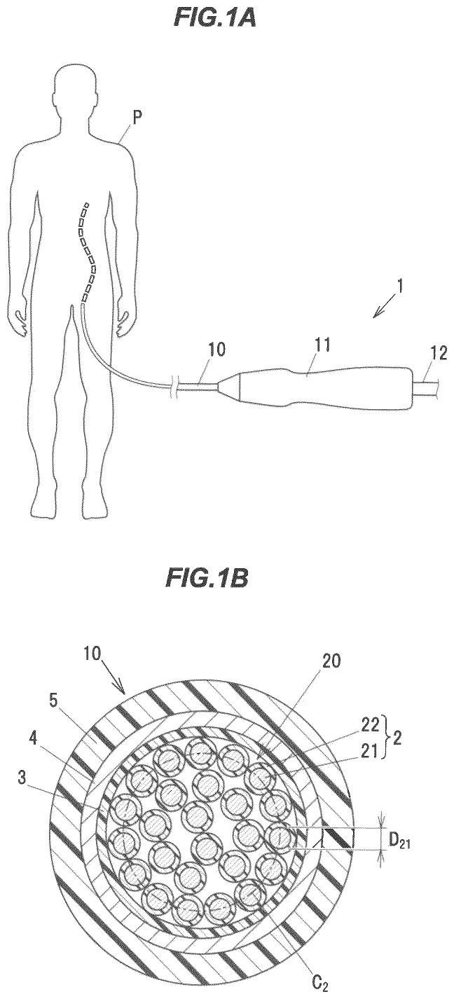

FIG. 1A is an explanatory diagram showing a usage state of a multi-electrode catheter 1 in which a multicore cable according to an embodiment of the present invention is used as a catheter cable 10. This multi-electrode catheter 1 is an example of a medical device using the multicore cable according to this embodiment. FIG. 1B is a cross-sectional view of the catheter cable 10.

The multi-electrode catheter 1 includes the catheter cable 10 and a handle 11 operated by an operator such as a surgeon. One longitudinal end of the catheter cable 10 is accommodated in the handle 11, and the other longitudinal end is inserted into the human body of the subject P for examination or treatment. In FIG. 1A, the portion of the catheter cable 10 inserted into the human body of the subject P is indicated by a dashed line.

As shown in FIG. 1B, the catheter cable 10 includes a plurality of insulated electric wires 2, a binder tape 3 wound around the plurality of insulated electric wires 2, a shield conductor 4 arranged around an outer periphery of the binder tape, and a tubular sheath 5 arranged around an outer periphery of the shield conductor 4. The plurality of insulated electric wires 2 are twisted together to form a wire bundle 20. The insulated electric wire 2 is a signal wire for transmitting electric signals. The sheath 5 is made of a fluorine resin such as PFA (copolymer of tetrafluoroethylene and perfluoroalkoxyethylene), and collectively covers the plurality of insulated electric wires 2 together with the binder tape 3 and the shield conductor 4.

In this embodiment, the catheter cable 10 has twenty-five insulated electric wires 2, and the insulated electric wires 2 are twisted in such a manner that specific insulated electric wires 2 among these insulated electric wires 2 will not always be present at a center portion or an outer peripheral portion of the wire bundle 20. However, the number of the insulated electric wires 2 in the catheter cable 10 is not limited thereto and may be, e.g., eight or more.

The insulated electric wire 2 includes a metal conductor wire 21 made of a high electrically conductive metal, and an insulating layer 22 covering the metal conductor wire 21. The metal conductor wire 21 is a single wire (i.e., solid wire) having a circular cross-section and is made of, e.g., copper or a copper alloy, or aluminum or an aluminum alloy. More specifically, soft copper alloy (e.g., annealed copper alloy) can be suitably used as the material of the metal conductor wire 21. A conductor diameter D₂₁ of the metal conductor wire 21 is, e.g., 0.010 mm or more and 0.085 mm or less.

The insulating layer 22 is made of thermosetting (heat curing) resin such as polyurethane, polyester, polyesterimide, polyamideimide, or polyimide. In the present embodiment, the insulated electric wire 2 is an enameled wire, and the insulating layer 22 is formed by applying a liquid resin material before curing to the outer periphery of the metal conductor wire 21 and then curing the applied resin by heating.

The multi-electrode catheter 1 is connected to a console (not shown) via a console cable 12 led out from the handle 11. The console is an information processing device equipped with a microprocessor, memory, etc., which amplifies a signal sent from the human body of the subject P via a plurality of insulated electric wires 2, and outputs an image signal for displaying the internal state of the human body of the subject P on the display. The handle 11 accommodates a substrate (i.e., board) having a plurality of pads to which the plurality of insulated electric wires 2 are connected. Signals sent from the human body of the subject P are relayed by this substrate and sent to the console by the console cable 12.

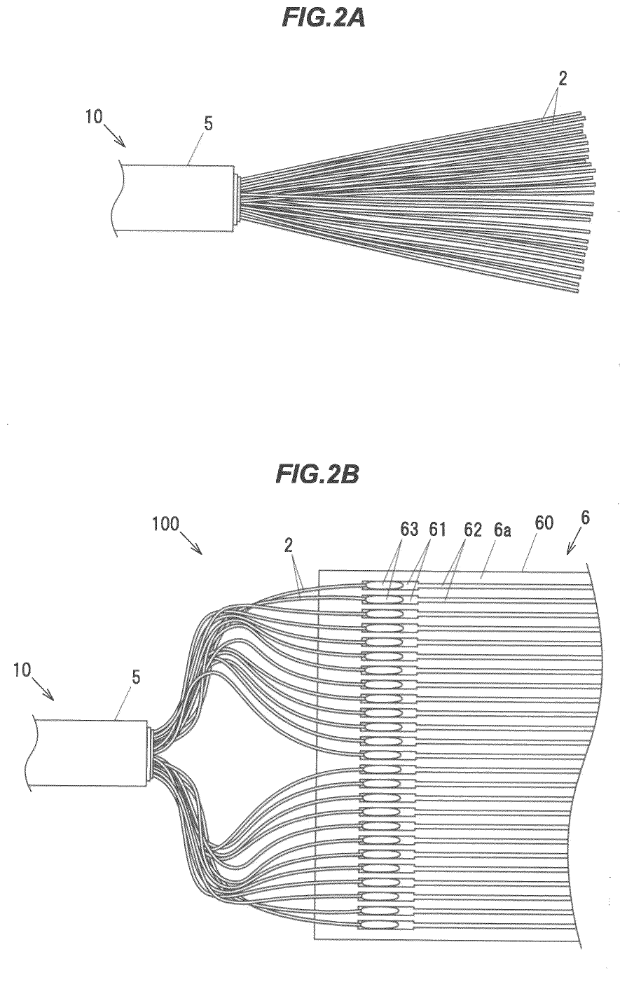

FIG. 2A is an explanatory diagram showing the cable end of the catheter cable 10 before the plurality of insulated electric wires 2 are connected to the substrate 6 within the handle 11. FIG. 2B is an explanatory diagram showing a state in which the plurality of insulated electric wires 2 are connected to the substrate 6.

The substrate 6 is one aspect of a terminal member to which the plurality of insulated electric wires 2 are connected at the end of the catheter cable 10. The catheter cable 10 and the substrate 6 constitute a multicore cable assembly 100. The catheter cable 10 has the sheath 5 removed over a predetermined length at its longitudinal end, and the plurality of insulated electric wires 2 extend from the end of the sheath 5. The binder tape 3 and the shield conductor 4 are cut off and removed near the end of the sheath 5.

The substrate 6 is an FPC (flexible printed circuit board), and has a plurality of electrode pads 61 and a plurality of wiring patterns 62 extending respectively from the electrode pads 61 on a surface 60a of a flexible plate-like base material 60. The insulating layer 22 is removed from the tip of the insulated electric wire 2 connected to the electrode pad 61 to expose the metal conductor wire 21, and the metal conductor wire 21 is electrically connected to the electrode pad 61. The electrode pad 61 has a rectangular shape when viewed in a direction perpendicular to the base material 60, and the metal conductor wire 21 extends over the electrode pad 61 along the long side direction of the rectangular shape.

The work of connecting the metal conductor wire 21 to the electrode pad 61 is performed manually by an operator, for example, under a magnifying glass or a microscope. FIG. 2B shows the case where the metal conductor wire 21 is connected to the electrode pad 61 by a solder 63, but the present invention is not limited thereto. The metal conductor wire 21 may be welded to the electrode pad 61 by an electrically conductive adhesive. Alternatively, the metal conductor wire 21 may be welded to the electrode pad 61 by, for example, ultrasonic waves.

When connecting the metal conductor wire 21 to the electrode pad 61, it is desirable from the viewpoint of workability that the insulated electric wire 2 extending from the sheath 5 is less undulated. Here, the term “undulation” (i.e., “waviness”) means that the insulated electric wire 2 bends irregularly when the bending tendency generated in the insulated electric wire 2 is released by removing the sheath 5 at the end of the cable. If the undulations of the insulated electric wire 2 are large, it is difficult to arrange the metal conductor wire 21 along the long side direction of the electrode pad 61 during the connection work, and short circuits or the like between the adjacent electrode pads 61 are likely to occur.

In the present embodiment, in order to reduce the bending tendency of the insulated electric wires 2 and to suppress the undulations of the insulated electric wires 2 at the portion extending from the sheath 5, each of the plurality of insulated electric wires 2 is plastically stretched in the longitudinal direction. The elongation rate of the insulated electric wire 2 by this plastic stretching is 0.5% or more and 10.0% or less. If the elongation rate is less than 0.5%, the effect of reducing the bending tendency of the insulated electric wire 2 will be small, and if the elongation rate is more than 10.0%, the wire disconnection of the metal conductor wire 21 will easily occur. The elongation rate of the insulated electric wire 2 is obtained by the arithmetic expression L₂/L₁, where L₁ is the unit length of the insulated electric wire 2 before plastic stretching, and L₂ is the length of the portion of the unit length L₁ after plastic stretching.

The contemplation that the bending tendency of the insulated electric wire 2 can be reduced by plastically stretching the insulated electric wire 2 was obtained by experiments by the present inventors. That is, when the conductor diameter of the metal conductor wire 21 is small, e.g., 0.1 mm or less, the outer peripheral surface of the metal conductor wire 21 is partly stretched due to the tensile stress that is unavoidably generated when the catheter cable 10 is manufactured. If this occurs, the insulated electric wire 2 will bend in such a manner that the portion where this elongation occurs will be on the outer peripheral side of the arc. However, by plastically stretching the entire insulated electric wire 2 in the longitudinal direction, the degree of variation in elongation at each part of the outer peripheral surface of the metal conductor wire 21 is reduced, and the bending tendency of the insulated electric wire 2 is reduced.

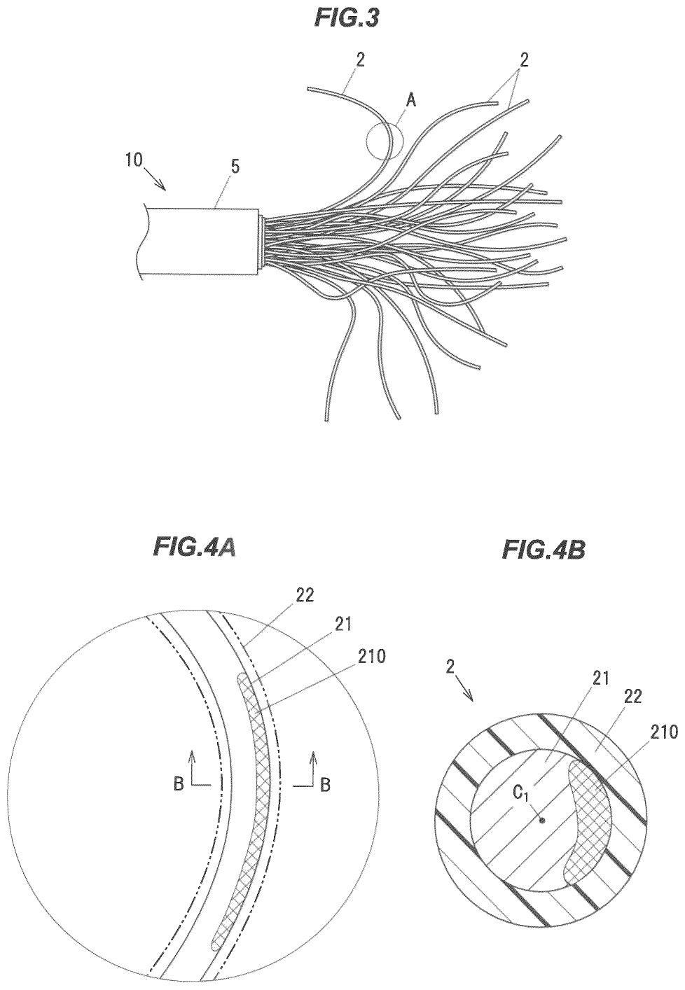

FIG. 3 is an explanatory view showing an example of the insulated electric wire 2 as a comparative example when the metal conductor wire 21 is not plastically stretched in the longitudinal direction. FIG. 4A is an explanatory view showing an enlarged portion A of FIG. 3. FIG. 4B is a cross-sectional view of the insulated electric wire 2 taken along a line B-B in FIG. 4A. In FIG. 4A, the insulating layer 22 is indicated by a phantom line, and the metal conductor wire 21 is indicated by a solid line. In addition, in FIGS. 4A and 4B, a portion 210 of the metal conductor wire 21 in which elongation due to tensile stress occurs is indicated by cross hatching.

As shown in FIGS. 4A and 4B, when elongation occurs in a part of the metal conductor wire 21 in the circumferential direction around the central axis C₁, the insulated electric wire 2 bends in such a manner that the portion 210 where the elongation occurs is placed on the outer circumference side of the arc. On the other hand, when the metal conductor wire 21 is plastically stretched in the longitudinal direction along the central axis C₁, the entire circumferential direction centered on the central axis C₁ is elongated, and the degree of variation in elongation is reduced, so that the bending tendency of the electric wire 2 is reduced.

In this embodiment, the insulated electric wire 2 is plastically stretched in the longitudinal direction at an elongation rate of 0.5% or more and 10.0% or less. Therefore, it is preferable to use a material having physical properties such as a tensile strength of 200 MPa or more and an elongation at break of 25% or more according to JIS C 3002 (Japanese Industrial Standards). By using a metal material with such physical properties for the metal conductor wire 21, it is possible to prevent the metal conductor wire 21 from breaking or the like even if the insulated electric wire 2 is plastically stretched in the longitudinal direction. For the catheter cable 10, it is preferable to use a soft copper alloy having the physical properties with the aforementioned range of tensile strength and the aforementioned range of elongation at break, and further an electrical conductivity of 70% or more. Note that non-annealed hard copper alloy may have, e.g., a high tensile strength of 700 MPa or more and a high electrical conductivity of 70% or more. However, the hard copper alloy should have an elongation at break of around 1%. It is not preferable to use the hard copper alloy since it will break at the limit of an elastic region and cannot be plastically stretched.

In this embodiment, the plurality of insulated electric wires 2 are plastically stretched by a method for manufacturing a catheter cable 10, which will be described later. A desirable twist rate of the plurality of insulated electric wires 2 is 0.5% or more and 5.0% or less. The twist rate K is obtained by the following formula, where P is a twist pitch of the insulated electric wire 2 and D is a pitch diameter of the wire bundle 20. Here, the pitch diameter D is a diameter of a circle (circle C₂ shown in FIG. 1B) passing through the center points of the plurality of insulated electric wires 2 in the outermost layer of the wire bundle 20 in the cross section orthogonal to the longitudinal direction of the catheter cable 10.

Moreover, it is desirable that the space factor of the wire bundle 20 is 70% or more, which is higher than the space factor of a general multicore cable. Appropriate plastic elongation can be generated in the insulated electric wires 2 by twisting and plastically stretching the plurality of insulated electric wires 2 in such a manner that the space factor of the wire bundle 20 is 70% or more. Here, the space factor is the ratio of the sum of the cross-sectional areas of the plurality of insulated electric wires 2 to the area of the circumscribed circle that includes the wire bundle 20 in the cross-section shown in FIG. 1B.

The elongation rate of the insulated electric wire 2 can be calculated from the difference in the length of the insulated electric wire 2 before and after plastic stretching as described above. The elongation rate of the insulated electric wire 2 can also be obtained from the difference in electrical resistance per the length of the insulated electric wire 2 before and after plastic stretching. This is because the cross-sectional area of the metal conductor wire 21 is reduced by stretching the insulated electric wire 2, and the electrical resistance is increased.

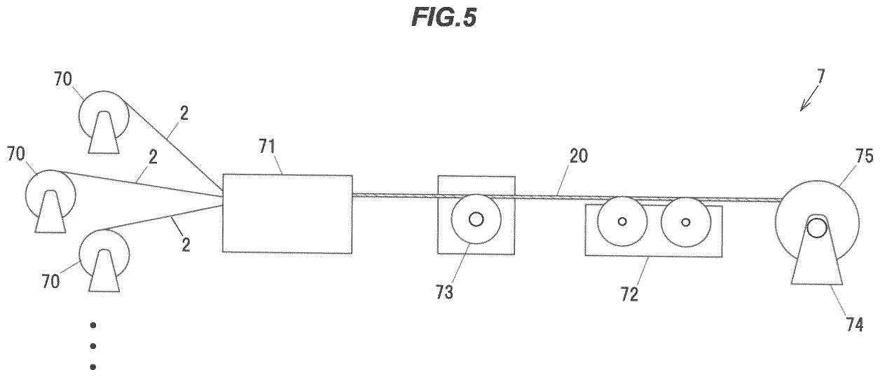

FIG. 5 is an explanatory view showing a configuration example of a manufacturing apparatus 7 for manufacturing the wire bundle 20 by twisting the plurality of insulated electric wires 2. The manufacturing apparatus 7 includes a wire twisting machine 71 for twisting the plurality of insulated electric wires 2 spirally, a plurality of supply reels 70 around which the plurality of insulated electric wires 2 supplied to the wire twisting machine 71 are respectively wound, and a take-up device 72 for taking up the wire bundle 20 in which the insulated electric wires 2 are twisted together from the wire twisting machine 71, a wire pulley (i.e., drawing wheel) 73 placed between the wire twisting machine 71 and the take-up device 72, and a winder 74 for winding the wire bundle 20 in which the plurality of insulated electric wires 2 are the plastically stretched.

The plurality of insulated electric wires 2 are stretched in the longitudinal direction in a plastic region by the tension generated between the take-up device 72 and the wire pulley 73 and wound by the winder 74 in the state of the twisted wire bundle 20 around a take-up drum 75. After that, the binder tape 3 is wound around the wire bundle 20, the shield conductor 4 is arranged around the binder tape 3, and the sheath 5 is formed around the shield conductor 4 by extrusion molding, whereby the catheter cable 10 is obtained. The elongation rate of the insulated electric wire 2 can be adjusted, for example, by increasing or decreasing the rotational resistance of the wire pulley 73.

If the insulated electric wire 2 can be plastically stretched at a predetermined elongation rate without arranging the wire pulley 73 between the wire twisting machine 71 and the take-up device 72, the wire pulley 73 can be omitted. Further, in place of the wire pulley 73, a plurality of wire pulleys for applying tension to the plurality of insulated electric wires 2 supplied to the wire twisting machine 71 and plastically stretching them may be placed between the supply reel 70 and the wire twisting machine 71.

Table 1 is a specification table showing the specifications of the catheter cable 10 according to one Example.

TABLE 1

| | |
|---|---|
| Number of insulated electric wires 2 | 25 |
| Outer diameter of insulated electric wire 2 before | 0.048 mm |
| plastic stretching | |
| Material of the insulating layer 22 | modified |
| | polyurethane |
| Thickness of insulating layer 22 before plastic | 0.008 mm |
| stretching | |
| Material of the metal conductor wire 21 | Copper alloy |
| Conductor diameter of metal conductor wire 21 | 0.032 mm |
| before plastic stretching | (Equivalent to |
| | AWG48) |
| Electrical resistance of metal conductor wire 21 | 29,091 Ω/km |
| before plastic stretching | |
| Twist rate | 1.0% |
| Electrical resistance of one metal conductor wire 21 | 29,382 Ω/km |
| per length of wire bundle 20 in which a plurality of | |
| insulated electric wires 2 that are not plastically | |
| stretched are twisted together (calculated value) | |
| Electric resistance of one metal conductor wire 21 | 30,374 Ω/km to |
| per length of wire bundle 20 in which a plurality of | 30,485 Ω/km |
| plastically stretched insulated electric wires 2 are | (Average |
| twisted together (measured value) | 30,423 Ω/km) |
| Elongation rate of insulated electric wire 2 | 3.4% to 3.8% |
| (calculated value) | (Average 3.5%) |

Here, the electrical resistance R₁ of one metal conductor wire 21 per length of the wire bundle 20 when the plurality of insulated electric wires 2 that are not plastically stretched are twisted together is obtained by multiplying the electrical resistance R₀ of the metal conductor wire 21 before plastic stretching by a coefficient corresponding to the twist rate K (i.e, 1+twist rate K (%)/100).

R₁=R₀×(1+K/100)

The elongation rate ε (%) of the insulated electric wire 2 is obtained by subtracting 1 from the quotient obtained by dividing the electric resistance R₂ per length of one metal conductor wire 21 when the plurality of insulated electric wires 2 that are plastically stretched are twisted together by the electrical resistance R₁ of one metal conductor wire 21 per length of the wire bundle 20 when the plurality of insulated electric wires 2 that are not plastically stretched are twisted together, and multiplying it by 100.

ε=(R₂/R₁−1)×100

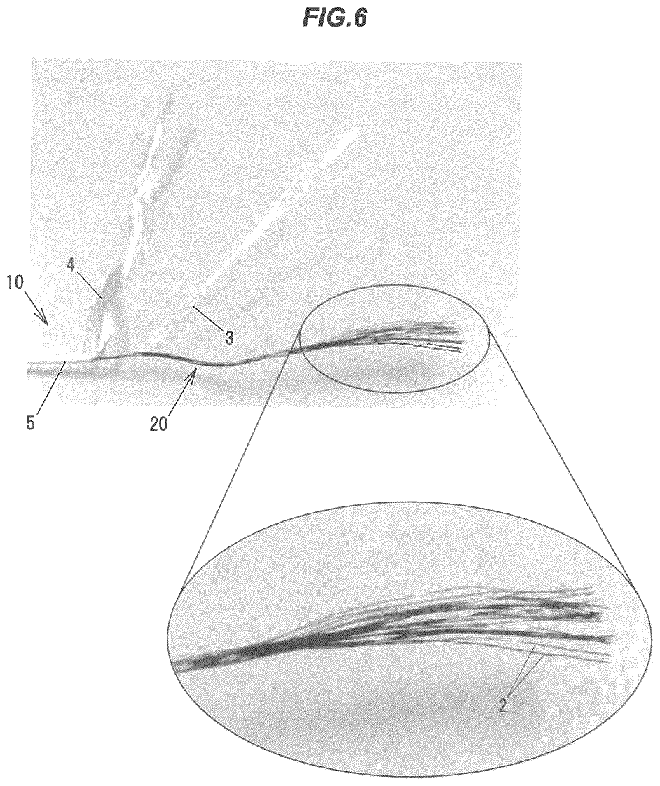

FIG. 6 is a photograph showing the catheter cable 10 according to the example having the above specifications. In FIG. 6, the sheath 5 at the end of the catheter cable 10 is removed, the binder tape 3 and the shield conductor 4 exposed from the end of the sheath 5 are removed from the wire bundle 20, and the twist of the plurality of insulated electric wires 2 at the tip of the wire bundle 20 are loosened (i.e., untwisted).

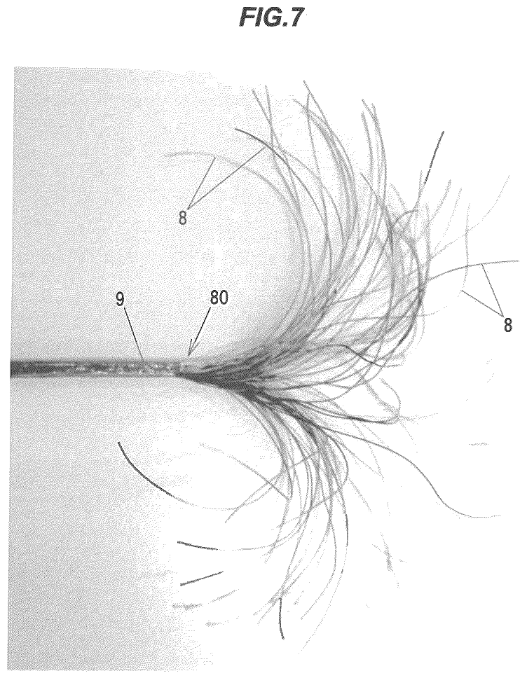

FIG. 7 is a photograph showing a plurality of insulated electric wires 8 that are not plastically stretched and a binder tape 9 wound around the outer circumferences of the plurality of insulated electric wires 8 in a comparative example. The plurality of insulated electric wires 8 are twisted together inside the binder tape 9 to form a wire bundle 80. The number of insulated electric wires 8 in the wire bundle 80 is different from the above Example.

As is clear from the comparison between FIGS. 6 and 7, in the catheter cable 10 in Example shown in FIG. 6, the undulations of the plurality of insulated electric wires 2 are sufficiently suppressed to the extent that they do not pose a problem in terminal processing.

### Effect of Embodiment

According to the embodiment described above, each of the plurality of insulated electric wires 2 is plastically stretched in the longitudinal direction, thereby reducing the bending tendency of each insulated electric wire 2. As a result, the workability of terminal processing of the catheter cable 10 is improved.

### Summary of Embodiment

Next, technical ideas understood from the embodiment described above will be described with reference to the reference numerals and the like in the embodiment. However, each reference numeral in the following description does not limit the constituent elements in the claims to the members and the like specifically shown in the embodiment.

According to the first feature, a multicore cable 10 (catheter cable 10) includes a plurality of insulated electric wires 2, each having a metal conductor wire 21 and an insulating layer 22 covering the metal conductor wire 21, wherein the plurality of insulated electric wires 2 are twisted together, wherein each of the plurality of insulated electric wires 2 is plastically stretched in a longitudinal direction at an elongation rate of 0.5% or more and 10.0% or less.

According to the second feature, in the multicore cable 10 as described in the first feature, in the plurality of insulated electric wires 2, the metal conductor wire 21 is a single wire with a circular cross-section, and the insulating layer 22 is composed of a thermosetting resin.

According to the third feature, in the multicore cable 10 as described in the second feature, in the plurality of insulated electric wires 2, the metal conductor wire 21 has a conductor diameter of 0.010 mm or more and 0.085 mm or less.

According to the fourth feature, in the multicore cable 10 as described in the first feature, the plurality of insulated electric wires 2 have a space factor of 70% or more.

According to the fifth feature, in the multicore cable 10 as described in the first feature, the plurality of insulated electric wires 2 have a twist rate of 0.5% or more and 5.0% or less.

According to the sixth feature, in the multicore cable 10 as described in the fifth feature, wherein the plurality of insulated electric wires 2 are twisted together in a number of eight or more.

According to the seventh feature, in the multicore cable 10 as described in the first feature, physical properties of the metal conductor wire 21 are a tensile strength of 200 MPa or more and an elongation at break of 25% or more.

According to the eighth feature, in the multicore cable 10 as described in the first feature, the plurality of insulated electric wires 2 are signal wires for transmitting electrical signals.

According to the ninth feature, the multicore cable 10 as described in the first feature, further includes a sheath 5 that collectively covers the plurality of insulated electric wires 2, and the plurality of insulated electric wires 2 extend from the sheath 5 at a cable end.

According to the tenth feature, a multicore cable assembly 100 includes the multicore cable 10 as described in any one of the first to ninth features, and a terminal member 6 (substrate 6) to which the plurality of insulated electric wires 2 are connected at an end of the multicore cable 10.

Although the embodiment of the present invention has been described above, the embodiment described above does not limit the invention according to the scope of claims. Also, it should be noted that not all combinations of features described in the embodiment are essential to the means for solving the problems of the invention.

In addition, the present invention can be modified appropriately and implemented. For example, in the above embodiment, the case where the substrate 6 is used as the terminal member to which the plurality of insulated electric wires 2 are connected is explained as an example. Further, a connector may be used as the terminal member, and electronic components such as IC may be used as the terminal member. Moreover, the multicore cable of the present invention can be used for various purposes other than the medical catheter cable 10.

1. A multicore cable, comprising:
a plurality of insulated electric wires, each comprising a metal conductor wire and an insulating layer covering the metal conductor wire,
wherein the plurality of insulated electric wires are twisted together,
wherein each of the plurality of insulated electric wires is plastically stretched in a longitudinal direction at an elongation rate of 0.5% or more and 10.0% or less.

2. The multicore cable, according to claim 1, wherein, in the plurality of insulated electric wires, the metal conductor wire is a single wire with a circular cross-section, and the insulating layer comprises a thermosetting resin.

3. The multicore cable, according to claim 2, wherein, in the plurality of insulated electric wires, the metal conductor wire has a conductor diameter of 0.010 mm or more and 0.085 mm or less.

4. The multicore cable, according to claim 1, wherein the plurality of insulated electric wires have a space factor of 70% or more.

5. The multicore cable, according to claim 1, wherein the plurality of insulated electric wires have a twist rate of 0.5% or more and 5.0% or less.

6. The multicore cable, according to claim 5, wherein the plurality of insulated electric wires are twisted together in a number of eight or more.

7. The multicore cable, according to claim 1, wherein physical properties of the metal conductor wire 21 are a tensile strength of 200 MPa or more and an elongation at break of 25% or more.

8. The multicore cable, according to claim 1, wherein the plurality of insulated electric wires are signal wires for transmitting electrical signals.

9. The multicore cable, according to claim 1, further comprising:
a sheath that collectively covers the plurality of insulated electric wires, and the plurality of insulated electric wires extend from the sheath at a cable end.

10. A multicore cable assembly, comprising:
the multicore cable according to claim 1; and
a terminal member to which the plurality of insulated electric wires are connected at an end of the multicore cable.
